(19) United States
(12) Patent Application Publication
(10) Pub. No.: US 2015/0238484 A1
(43) Pub. Date: Aug. 27, 2015

(54) Use of Flibanserin for the Treatment of Sexual Disorders in Females

(71) Applicant: SPROUT PHARMACEUTICALS, INC., Raleigh, NC (US)

(72) Inventor: Robert Pyke, Ridgefield, CT (US)

(21) Appl. No.: 14/583,292

(22) Filed: Dec. 26, 2014

Related U.S. Application Data
(63) Continuation of application No. 13/671,864, filed on Nov. 8, 2012, now abandoned.
Application No. 13/671,864 is a continuation of application No. 12/306,945, filed on Feb. 9, 2009, now abandoned.
(60) Provisional application No. 60/830,988, filed on Jul. 14, 2006.

(51) Int. Cl. A61K 31/496

(52) U.S. Cl. CPC A61K 31/496

(57) ABSTRACT

The invention is directed to the use of flibansenn or a pharmacologically acceptable derivative thereof, for the preparation of a medical composition For lbe treatment of Sexual Disorder in females whereby the medication of a patient is selected to achieve a significant change (with administration of a therapeutically effective amount of flihanserin) starting from a baseline (without administration of flibanserin), the significant change being achieved within at least one primary criteri-a for efficacy and optionally within at least one secondary criteria of efficacy.

### FIELD OF THE INVENTION

The present invention is directed to the novel use of Flibanserin for the preparation of a medicament for the treatment of Sexual Disorders in females.

### BACKGROUND OF THE INVENTION

Female Sexual Dysfunctions or Disorders (also abbreviated as “FSDs”) is highly prevalent and it is estimated that about 20 to 50% of the women are affected. Thus there exists a need for the development of novel therapies which are adapted to the problems of each individual case.

The compound 1-[2-(4-(3-trifluoromethyl-phenyl)piperazin-1-yl)ethyl]-2,3-dihydro-1H-benzimidazol-2-one (flibanserin) is disclosed in form of its hydrochlorid in European Patent Application EP-A-526434 and has the following chemical structure:

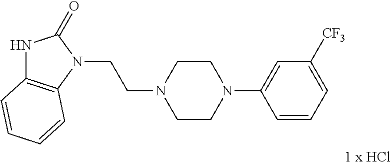 [SMILES: O=c1[nH]c2ccccc2n1CCN1CCN(c2cccc(C(F)(F)F)c2)CC1]

Flibanserin shows affinity for the 5-HT_{1A} and 5-HT₂-receptor. It is therefore a promising therapeutic agent for the treatment of a variety of diseases, for instance depression, schizophrenia, and anxiety.

According to WO 03/035072 A1 it is known to use flibanserin in the treatment of Sexual Desire Disorders; in WO 20051102343 A1 it is described to use flibanserin in the treatment of premenstrual and other female Sexual Disorders.

However, the known use of Flibanserin has the disadvantage for not being adapted to each individual case and a suitable concept to optimize the therapy is missing. Furthermore the known use of flibanserin has the disadvantage that sometimes mild or moderate symptoms such as sedation may occur.

Furthermore, there is a paucity on research on Sexual Disorders and Sexual Dysfunctions consequently the physiological underpinnings of this disorders are currently not totally understood. To date there are no approved pharmacological treatments since clinical development and off-label usage has focused mainly on hormone products.

Therefore, it is an object of the present invention to select and optimize the use of flibanserin for the treatment of Sexual Disorders for each and every individual type of Sexual Disorder or Sexual Dysfunction in women.

### DESCRIPTION OF THE INVENTION

Surprisingly, it has been found that it is possible to establish a correlation between different types of specific testing criteria and the use of flibanserin suitable for the treatment of Sexual Disorders which assists to successfully treat the related diseases.

Therefore, the present invention provides the use of flibanserin or a pharmacologically acceptable derivative thereof for the preparation of a medical composition for the treatment of Sexual Disorder in females whereby the medication of a specific patient to be treated is adjusted or selected to achieve a significant change (with administration of a therapeutically effective amount of flibanserin) starting from a baseline (without administration of flibanserin), the significant change being achieved within at least one primary criteria for efficacy,

the primary criteria for efficacy being selected from the group consisting of criteria a) and/or criteria b),

whereby

criteria a) is the number of satisfying sexual events (and frequency of sexual activity and orgasms), which is determined with the following algorithm:

total monthly events=28×(sum of the number of events)/(sum of number of days entered)

and

criteria b) is the level of sexual desire collected daily, which is determined with the following algorithm:

Desire days=28×(number of entries with moderate or strong desire)/(number of entries)

the significant change determined by comparing the algorithm of criteria a) with and without administration of flibanserin is represented by a satisfying sexual events increase of at least two;

and/or

the significant change determined by comparing the algorithm of criteria b) with and without administration of flibanserin is represented by a desire days increase of at least four per month.

Therefore, the present inventors have developed a concept of two primary criteria and optionally a number of secondary criteria, which allows to select the optimized therapy and e.g. dose or administration mode, for each individual patient. As a result, at least one of the two primary criteria a) and b) should meet a lower limit as above-defined in order to have the optimum therapeutic success. The two primary criteria are identified as criteria a) and criteria b), whereby the criteria a) (also referred to as “first co-primary endpoint”) represents the change from a baseline, in the frequency of satisfying sexual events of said one patient to be treated. The satisfying sexual events are registered by the respective patient herself and for example written down in a book or booklet, for example, entered in a diary, particularly an electronic diary. Sexual events include sexual intercourse, oral sex, masturbation or genital stimulation by the partner.

The other “co-primary endpoint”, i.e. criteria b), is the change from a baseline in the monthly sum of responses to the daily desire question, preferably determined and written down by the respective patient herself, for example in a booklet such as a diary, particularly a notebook or computer in form of an electronic diary. An electronic diary is preferably used, i.e. a diary designed in a personal computer or a small personal handheld device which may be used by the patient to record the information daily. But the information may be also collected in handwritten form in a map or the like.

The baseline of both primary criteria represents an estimation or evaluation of the respective person or female patient to be treated of her sexual life. In other words patient's own assessment of the sexual life in a status without administration of flibanserin is determined to be the baseline, i.e. the baseline is assumed to represent no effect (“null line”), i.e. the patient being not treated with any medicament containing flibanserin. For example the baseline, for criteria a) is the above-mentioned algorithm according to which applies:

total monthly events=28×(sum of the number of events)/(sum of number of days entered)

wherein the total monthly events are determined by the female patient, for example, over a defined period of time, e.g. 4 weeks up to several months in the past of the patient. The judgment of whether the event is satisfying or not is made by the patient herself. It is a matter of course that adverse effects which could distort the outcome should be excluded as much as possible.

Subsequently the treatment with a therapeutically amount of flibanserin is started and during the treatment the patient makes again an estimation or evaluation of her sexual life. In other words patient's own assessment of the sexual life in a status with administration of flibanserin is determined according to the above given algorithm for criteria a) and/or criteria b) for efficacy. A comparison between both states with and without administration of flibanserin in at least one criteria performed for said patient serves to modify the therapy accordingly in order to meet the above-described requirements for the crirteria of efficacy as defined. Said procedure may be repeated for a number of times based on the above in order to further optimize the therapy.

In order to explain the present invention a simplified example is given as follows:

baseline:
patient without administration of flibanserin:
sum of number of events=2 (given by the patient)
sum of number of days entered=28
It follows the total monthly events=28×2/28=2

That is the baseline for the total monthly events represents in this case 2 for this specific patient.

Subsequently the flibanserin therapy is started and after a defined period of time, for example 1 month, the above-given algorithm is checked again for the same patient. For example

the patient with administration of flibanserin:
sum of number of events=4 (given by the same patient as above)
sum of number of days entered=28
It follows the total monthly events=28×2/28=4

Therefore the change determined by comparing the algorithm of criteria a) with and without administration of flibanserin (4−2=2) represents for said patient in the above example a significant change according to the present invention wherein the significant change is represented by a satisfying sexual events increase of at least two. Consequently, criteria a) for efficacy is fulfilled. The therapy of this patient is successful. The dose is optimized based on the primary criteria to successfully treat said specific patient.

The same findings as for the primary criteria a) apply for the primary criteria b).

If said change is too low, i.e. the change is lower than the above given limit, the change is not significant, the therapy must be modified, e.g. the dose could be raised accordingly, until the above lower limit is adjusted or exceeded. That is the medication or dose administered to a patient is adjusted correspondingly to at least one of the primary criteria for efficacy to achieve the maximum possible therapeutic effect and determine the most effective dose for the individual patient.

Preferably criteria a) and criteria b) are fulfilled at the same time, i.e. at least the above-mentioned lower limits are both achieved simultaneously. Therefore, it is preferred that a satisfying sexual events increase of at least two (criteria a)) and a desire days increase of at least four per month (criteria b)) are fulfilled.

As a result, the comparison of the levels or changes reached when the patient is treated with flibanserin and the levels when the patient is not treated with flibanserin offers to optimize therapy to result in an individually tailored therapy.

In addition to the two primary criteria for efficacy, the present inventors have developed several secondary criteria for efficacy (also referred to as “secondary endpoints”) which optionally could be taken into account.

One secondary criteria being the key secondary criteria (“key secondary endpoint”) is the Female Sexual Distress Scale (FSDS) or the Female Sexual Distress Scale-Revised (FSDS-R) test (optional criteria c) for efficacy) which quantifies the change in the personal distress due to Sexual Dysfunction or Disorder. As above explained it is compared a baseline, that is the estimation of the female patient without the treatment of flibanserin (the score achieved in the test) e.g. over a defined period of time, and the level reached after a defined period of time with treatment of a therapeutically effective amount of flibanserin (the score achieved in the test). The change from the baseline in the FSDS or FSDS-R score, respectively, gives a further indication about the therapy. According to the present invention a 12-item questionnaire is the FSDS and an additional question 13 (FSDS plus question 13, 13-item questionnaire) represents the FSDS-R test. The maximum score of the FSDS-R indicating a maximum level of sexual distress is 52. Both tests are shown in the experimental section.

The baseline for the FSDS or FSDS-R test is represented by the untreated patient (the score achieved in the test). The change is determined with the patient treated with a medicament containing flibanserin. That is the score of the untreated and the treated patient are compared and in case there is an improvement (improvement with regard to the score compared between a status without treatment of flibanserin and a status with treatment of flibanserin) the therapy is regarded to be optimized for this criteria for efficacy.

Other secondary endpoints or criteria for efficacy according to the present invention are:
The Female Sexual Functioning Index (FSFI) (optional criteria d) for efficacy) is a self-administered questionnaire to assess FSD that consists of 19 questions that are scored from “0” to “5”. The scale contains six domains:

desire, arousal, lubrication, orgasm, satisfaction and pain. The total score is a weighted average of the six domains each contributing a maximum of “6” points to the total, so the maximum score of FSFI is “36”. The questionnaire allows to establish changes from baselines on the FSFI total score and individual domains. An improvement of the test results indicates that the therapy is more successful. The FSFI is shown in the practical section.
The Patient Global Impression (PGI) of Improvement (optional criteria e) for efficacy) is a simple evaluation completed by the patient to assess the patient's overall evaluation of her status. The PGI of Improvement is rated ordinally from one to seven. The mean scores on the PGI improvement may be used. A PGI of Improvement of at least ‘1’, preferably at least ‘2’ shows a more optimized overall therapy. The questionnaire for the Patient Global Impression (PGI) of Improvement is shown in the practical section.
The Patient Benefit Evaluation (optional criteria f) for efficacy) is a single question asking the patient whether or not she experienced a meaningful benefit from the therapy. The question may be “Overall, do you believe that you have experienced a meaningful benefit from the medication or therapy?” An improvement in this test performed brings about a still more optimized therapy.

Therefore, the present invention provides an on-treatment efficacy assessment, which is an efficacy assessment that occurred between the first dose and during the treatment in order to adjust the optimum dose level and/or administration mode etc. for each type of Sexual Disorder.

Therefore, the medication of a flibanserin therapy to be employed is adjusted in accordance with criteria a) and/or b) for efficacy, and optionally criteria c), d), e) and/or f), to achieve the most effective therapy and dose of flihanserin for the individual female patient to be treated.

The disease or illness to be treated in the present invention are Female Sexual Disorders or Dysfunctions. The generic term “Sexual Disorders” or “Sexual Dysfunctions” according to the present invention shall be understood within its broadest meaning and shall include all kind of Sexual Disorders and Dysfunctions known. “Sexual disorders” or “Sexual Dysfunctions”, both expressions being virtually used synonymously in the present invention and may be characterized by a disturbance in sexual desire, in the physiological changes that characterize the sexual response cycle or by pain associated with sexual intercourse. The sexual response cycle may be divided in the phases Desire, Excitement, Orgasm and Resolution and the disorders of sexual response may occur at one or more of these phases, multiple disorders or dysfunctions may be present. Sexual disorders may cause distress and personal difficulty and may be associated with other disorders such as mood disorders or anxiety disorders (Obsessive-Compulsive Disorder, Panic Disorder with agoraphobia and specific Phobia) (see Diagnostic and Statistical Manual of Mental Disorders, 4th edition, Text Revision. Washington D.C., American Psychiatric Association, 2000).

Sexual Disorders and/or Sexual Dysfunctions (hereinafter simply referred to as “Sexual Disorder(s)” or “Disorder(s)”) are categorized into several main types which may be further divided in several subtypes all of which are included herein.

Examples of Sexual Disorders are Sexual Desire Disorders, (i.e., Hypoactive Sexual Desire Disorder, Sexual Aversion Disorder), Sexual Arousal Disorders (i.e., Female Sexual Arousal Disorder, Male Erectile Disorder), Orgasmic Disorders (i.e., Female Orgasmic Disorder, Male Orgasmic Disorder, Premature Ejaculation). Sexual Pain Disorders (Dyspareunia, Vaginismus, Noncoital Pain Disorder), Sexual Dysfunction due to a General Medical Condition, Substance-induced Sexual Dysfunction, and Sexual Dysfunction not otherwise specified (cf. Diagnostic and Statistical Manual of Mental Disorders, ibid.).

According to Diagnostic and Statistical Manual of Mental Disorders, 4th edition, Text Revision, Washington D.C., American Psychiatric Association, 2000, the disclosure thereof being incorporated in the present specification by reference, Hypoactive Sexual Desire Disorder (HSDD) is characterized by a general loss of sexual desire leading to distress. HSDD may be in detail defined to be a deficiency or absence of sexual fantasies and desire for sexual activity (criterion A) whereby the dysfunction must cause marked distress or interpersonal difficulties (criterion B) and the Sexual Disorder is not better accounted for by another disorder (criterion C).

Sexual Aversion Disorder is defined as a persistent or recurrent extreme aversion to, and avoidance of, all or almost all genital sexual contact with a sexual partner. Sexual Arousal Disorder is characterized to be a persistent or recurrent inability to attain, or maintain until completion of the sexual activity. Orgasmic Disorders is a persistent or recurrent delay in, or absence of, orgasm following normal sexual excitement phase. Sexual Pain Disorders is related with genital pain which may be associated with sexual intercourse or the involuntary contraction of the perineal muscles surrounding the outer third of the vagina.

Sexual Dysfunction due to a General Medical Condition may be determined based on history, laboratory findings or physical examination that the Sexual Disorder is fully explained by direct physiological effects of a general medical condition.

Further, Substance-induced Sexual Dysfunction may be a disorder or dysfunction exclusively caused by the physiological effects of a drug abuse, a medication or toxin exposure. It depends on the type or amount of the substance used or the duration of use or exposure.

The Sexual Dysfunction not otherwise specified includes Sexual Dysfunctions that do not meet criteria for any other specific Sexual Dysfunction.

It should be noted that the definitions developed for the categorization of Sexual Disorders have no fixed or exact limits, but transitions and/or overlappings may be possible. One type of Sexual Disorder may also occur in association with other Sexual Disorders or Sexual Dysfunctions. Then, for example, the predominant Sexual Disorder is selected, the type of disorder assigned is not better accounted for by another type of disorder.

The subtypes in order to further categorize Sexual Disorder indicate the onset (lifelong type and acquired type), context (generalized type and situational type) and etiological factors (due to psychological factors and due to combined factors) associated with the Sexual Disorder. These subtypes do not apply to a diagnosis of Sexual Dysfunction due to a general medical condition or Substance-induced Sexual Dysfunction.

The “lifelong type” refers to such Sexual Disorders of the present invention, which have been present since the onset of sexual functioning. The “acquired type” applies to such Sexual Disorders of the present invention which developed only after a period of normal sexual functioning. The “generalized type” refers to such Sexual Disorders of the present invention wherein the disorder is not limited to certain types of stimulation, situations, or partners. The “situational type” applies to such Sexual Disorders of the present invention wherein the disorder is limited to certain types of stimulation, situations, or partners. The subtype due to “psychological factors” applies when psychological factors are judged to have the major role in the onset, severity, exacerbation, or maintenance of the Sexual Disorder, and general medical conditions and substance play no role in the etiology of the Sexual Disorder. Finally the subtype due to “combined factors” applies when 1) psychological factors are judged to have a role in the onset, severity, exacerbation, or maintenance of the Sexual Disorder, and 2) a general medical condition or substance use is also judged to be contributory but is not sufficient to account for a Sexual Disorder (cf. Diagnostic and Statistical Manual of Mental Disorders, 4th edition, Text Revision. Washington D.C., American Psychiatric Association, 2000).

In studies of female patients suffering from Sexual Disorders it has been found that flibanserin, optionally in form of the free base, as well as a pharmacologically acceptable derivative such as the pharmacologically acceptable acid addition salts and/or optionally the hydrates and/or solvates thereof has a positive effect on Sexual Disorders and/or Dysfunctions for example it shows sexual desire enhancing properties.

Furthermore, Sexual Desire Disorders and/or Dysfunctions may be treated in female patients being in pre-menopausal or post-menopausal status. In other words the above mentioned types of Sexual Desire Disorders may be also treated in pre-menopausal or post-menopausal women,

Also premenstrual disorders should be included in Sexual Disorders, for example Premenstrual Disorders selected from the group consisting of Premenstrual Dysphoria, Premenstrual Syndrome, Premenstrual Dysphoric Disorder. Premenstrual and other Sexual Disorders are described in WO 2005/102343 the whole disclosure thereof being incorporated into the present specification by reference.

Accordingly, the present invention relates to the use of flibanserin, optionally in form of the free base, the pharmacologically acceptable acid addition salts and/or the hydrates and/or solvates thereof for the preparation of a medicament for the treatment of Sexual Disorder selected from the group consisting of Sexual Desire Disorders, (i.e., Hypoactive Sexual Desire Disorder, Sexual Aversion Disorder), Sexual Arousal Disorders Female Sexual Arousal Disorder, Male Erectile Disorder), Orgasmic Disorders (i.e., Female Orgasmic Disorder, Male Orgasmic Disorder, Premature Ejaculation). Sexual Pain Disorders (Dyspareunia, Vaginismus, Noncoital Pain Disorder), Sexual Dysfunction due to a General Medical Condition, Substance-Induced Sexual Dysfunction, and Sexual Dysfunction not otherwise specified, the treatment being adapted to the individual patient as defined above with regard to the primary and secondary criteria for efficacy.

Disorders of sexual desire are, for example, described in WO 03/035072 A1, the whole disclosure thereof being incorporated into the present specification by reference.

Particular preferred according to the invention is the use of flibanserin, optionally in form of the free base, the pharmacologically acceptable acid addition salts and/or the hydrates and/or solvates thereof for the preparation of a medicament for the treatment of Sexual Disorder selected from the group consisting of Sexual Desire Disorder, preferably Hypoactive Sexual Desire

Disorder, Sexual Aversion Disorder, loss of sexual desire, lack of sexual desire, decreased sexual desire, inhibited sexual desire, loss of libido, libido disturbance, and frigidity, the treatment being adapted to the individual patient as defined above with regard to the primary and secondary criteria for efficacy.

In a particularly preferred embodiment the invention relates to the use of flibanserin, optionally in form of the free base, the pharmacologically acceptable acid addition salts and/or the hydrates and/or solvates thereof for the preparation of a medicament for the treatment of Sexual Disorder selected from the group of Hypoactive Sexual Desire Disorder and loss of sexual desire, the treatment being adapted to the individual patient as defined above with regard to the primary and secondary criteria for efficacy.

The beneficial effects of flibanserin can be observed in all kind of Sexual Disorders of female and male patients independent of the main type or subtype, independent of the onset, context and etiological factors associated with the Sexual Disorder. That is regardless of whether the Sexual Disorder existed lifelong or was acquired, or independent of context and etiologic origin, the treatment will be possible.

In a further preferred embodiment the invention relates to the use of flibanserin, optionally in form of the free base, a pharmacologically acceptable acid addition salt and/or a hydrate and/or a solvate thereof for the preparation of a medicament for the treatment of Sexual Disorder selected from the group consisting of acquired Hypoactive Sexual Desire Disorder, acquired Sexual Aversion Disorder, acquired loss of sexual desire, acquired lack of sexual desire, acquired decreased sexual desire, acquired inhibited sexual desire, acquired loss of libido, acquired libido disturbance, and acquired frigidity, the treatment being adapted to the individual patient as defined above with regard to the primary and secondary criteria for efficacy.

Furthermore the present invention relates to the generalized or situational subtype of any of the above mentioned conditions and/or to such which are due to psychological factors or due to combined factors).

Therefore the term “acquired Hypoactive Sexual Desire Disorder” etc. refers to Hypoactive Sexual Desire Disorder in women, which developed after a period of normal sexual functioning.

The present invention is also directed to the use of flibanserin or a pharmacologically acceptable derivative thereof, for the preparation of a medical composition for the treatment of Sexual Disorder in females, characterised in that the dose is administered once daily or twice daily, preferably once daily.

Preferably, the dose is administered to a patient in the morning and the evening, more preferably once in the morning and once in the evening, most preferably once in the evening only consecutively over a period of time.

According to a preferred embodiment of the present invention the first dose of a beginning therapy is administered to a patient in the evening. As a result side-effects such as sedation are of lesser significance.

The dosis range of flibanserin applicable per day is between 0.1 to 100 mg. However, due to the severeness of the Sexual Disorder it is selected a low dose range, a lower medium dose range, an upper medium dose range and a higher dose range of flibanserin, which are preferably defined as follows:

a low dose range is preferably 10 to 40 mg, more preferably 15 to 35 mg, particularly about 25 mg;
a lower medium dose range is preferably 41 to 60, more preferably 45 to 55 mg, particularly about 50 mg;
an upper medium dose range is preferably 61 to 90, more preferably 65 to 85 mg, particularly about 75 mg; and
a high dose range is preferably 91 to 100 mg, particularly about 100 mg.

According to the present invention the dose is preferably administered to a patient in a low dose range and/or lower medium dose and/or upper medium dose once daily or twice daily, or in a high dose range once daily, more preferably the dose is administered to a patient once daily every evening. The medication is, for example, consecutively over a period of time.

With regard to an upper limit of dose administered to a patient a maximum dose is preferably of at most 100 mg/day, more preferably at most 90 mg/day, most preferably at most 80 mg/day.

Preferably, the dose is administered to a patient in the morning and the evening, more preferably once in the morning (25 or 50 mg of flibanserin) and once in the evening (25 or 50 mg of flibanserin), most preferably once in the evening only (50 or 100 mg of flibanserin) consecutively over a period of time.

Preferably, the above defined doses may be used as a basis for the above-defined optimized treatment being adapted to the individual patient using at least on of the primary and optionally at least one of secondary criteria for efficacy as described.

Flibanserin can be used in form of the free base, or in form of any known pharmacologically acceptable derivative thereof such as its pharmaceutically acceptable acid addition salts and/or optionally in form of the hydrates and/or solvates thereof. Suitable acid addition salts include for example those of the acids selected from succinic acid, hydrobroniic acid, acetic acid, fumaric acid, maleic acid, methanesulphonic acid, lactic acid, phosphoric acid, hydrochloric acid, sulphuric acid, tartaric acid and citric acid. Mixtures of the abovementioned acid addition salts may also be used. From the aforementioned acid addition salts the hydrochloride and the hydrobromide, particularily the hydrochloride, are preferred.

If flibanserin is used in form of the free ba e it is preferably used in form of flibanserin polymorph A which represents the free base of flibanserin in a specific polymorphic form. Polymorph A and a process for its preparation are disclosed in WO 03/014079 A1, the whole disclosure thereof being incorporated by reference into the present specification.

Flibanserin, optionally used in form of the free base, the pharmacologically acceptable acid addition salts and/or the hydrates and/or solvates thereof, may be incorporated into the conventional pharmaceutical preparation in solid, liquid or spray form. The composition may, for example, be presented in a form suitable for oral, rectal, parenteral administration or for nasal inhalation: preferred forms include for example capsules, tablets, coated tablets, ampoules, suppositories, and nasal spray.

The active ingredient may be incorporated in one or more excipients, one or more carriers, one or more diluents, one or more vehicles, e.g. aqueous or non aqueous, and/or one or more additives, conventionally used in pharmaceutical compositions such as, for example, talc, gum arabic, lactose, gelatine, magnesium stearate, corn starch, polyvinyl pyrrolidone, semisynthetic glycerides of fatty acids, benzalconium chloride, sodium phosphate, EDTA, and polysorbate 80. The compositions are advantageously formulated in dosage units, each dosage unit being adapted to supply a single dose of the active ingredient. Depending from the administration form the ingredients are selected accordingly.

Suitable tablets may be obtained, for example, by mixing the active substance(s) with known excipients, for example inert diluents such as calcium carbonate, calcium phosphate or lactose, disintegrants such as corn starch or alginic acid, binders such as starch or gelatine, lubricants such as magnesium stearate or talc and/or agents for delaying release, such as carboxymethyl cellulose, cellulose acetate phthalate, or polyvinyl acetate. The tablets may also comprise several layers.

Coated tablets may be prepared accordingly by coating cores produced analogously to the tablets with substances normally used for tablet coatings, for example collidone or shellac, gum arabic, talc, titanium dioxide or sugar. To achieve delayed release or prevent incompatibilities the core may also consist of a number of layers. Similarly the tablet coating may consist of a number of layers to achieve delayed release, possibly using the excipients mentioned above for the tablets.

Syrups or elixirs containing the active substances or combinations thereof according to the invention may additionally contain a sweetener such as saccharine, cyclamate, glycerol or sugar and a flavour enhancer, e.g. a flavouring such as vanilline or orange extract. They may also contain suspension adjuvants or thickeners such as sodium carboxymethyl cellulose, wetting agents such as, for example, condensation products of fatty alcohols with ethylene oxide, or preservatives such as p-hydroxybenzoates.

Solutions for injection are prepared in the usual way, e.g. with the addition of preservatives such as p-hydroxybenzoates, or stabilisers such as alkali metal salts of ethylenediamine tetraacetic acid, and transferred into injection vials or ampoules.

Capsules containing one or more active substances or combinations of active substances may be prepared for example by mixing the active substances with inert carriers such as lactose or sorbitol and packing them into gelatine capsules.

Suitable suppositories may be made for example by mixing with carriers provided for this purpose, such as neutral fats or polyethyleneglycol or the derivatives thereof.

The advantages of the present invention are manifold:

The use of flibanserin according to the present invention is optimized in the sense that the treatment regimen is specifically tailored to the individual patient suffering from FSD.

The inventive concept does give a dear indication for the therapy, e.g. administration mode, and achieves significant benefits to the patient being treated. The tailored treatment of the present invention may assist to reduce the dose to the minimum dose necessary to obtain the best therapeutically positive effects in the sense of a meaningful therapeutic response.

The therapy offers reduced side effects due to the optimized medication. The use according to the present invention achieves the maximum possible therapeutic effect by providing the best individual treatment schedule and complies with the specific needs of a patient.

The developed primary and secondary criteria and lower limits given provide clear evidence of optimized treatment for FSD, particular HSDD, by the use of a minimized dosemaximized effect correlation.

### Practical Section

In the following Practical Section the above-described tests are given in detail.

1. A method for treating a sexual disorder in a female patient comprising administering a medication comprising flibanserin or a pharmacologically acceptable derivative thereof whereby the medication of a the female patient is selected to achieve a significant change (with administration of a therapeutically effective amount of flibanserin) stalling from a baseline (without administration of flibanserin),
wherein the significant change is within at least one primary criteria for efficacy, the primary criteria for efficacy being selected from the group consisting of criteria a) and criteria b),
wherein:
criteria a) is the number of satisfying sexual events determined with the following algorithm:
total monthly events=28×(sum of the number of events)/(sum of number of days entered) And
criteria b) is the level of sexual desire collected daily, which is determined with the following algorithm:
Desire days=28×(number of entries with moderate or strong desire)/(number of entries)
wherein the significant change determined by comparing the algorithm of criteria a) with and without administration of flibanserin is represented by a satisfying sexual events increase of at least two per month; and
the significant change determined by comparing the algorithm of criteria b) with and without administration of flibanserin is represented by a desire days increase of at least four per month.

2. The method for treating a sexual disorder in a female patient according to claim 1, whereby the medication of i4 the female patient is selected to achieve a significant change (with administration of a therapeutically effective amount of flibanscrin) starting from a baseline (without administration of flibanserin), wherein the significant change is within at least one secondary criteria for efficacy,
whereby the secondary criteria for efficacy are criteria c), criteria d), criteria e), and/of criteria f),
wherein:
criteria c) is the Female Sexual Distress Scale (FSDS) or the Female Sexual Distress Scale-Revised (FSDS-R) test;
criteria d) is the Female Sexual Functioning Index (FSFI);
criteria e) is the Patient Global Impression (PGI) of Improvement; and
criteria is the Patient Benefit Evaluation,
wherein the significant change determined by comparing at least one of the criteria c) to f) with and without administration of tlibanserin, respectively, is represented by
in criteria c) a PGI of Improvement of at least ‘1’, preferably at least ‘2’; and/or
in criteria d) an improvement of the result(s) of the test performed; and
in criteria e), an improvement of the result(s) of the test performed; and
in criteria f), an improvement of the result(s) of the test performed.

3. The method for treating a sexual disorder in a female patient according to claim 1, wherein the significant change of at least one primary criteria for efficacy is determined over aperiod of time of one month or longer.

4. The method for reatin a sexual disorder in a female patient according to claim 1, wherein he medication to be administered to the female patient is adjusted to have a significant change in at least one of criteria a) or b) to achieve the therapeutic most effective dose and/or most effective administration mode of the medication.

5. A method for treating a sexual disorder in a female patient comprising administering a medication comprising flibanserin or a pharmacologically acceptable derivative thereof wherein the dose is administered once daily consecutively over a period of time.

6. A method for treating a sexual disorder in a female patient according to claim 1, wherein the dose is administered in the morning and the evening.

7. The method for treating a sexual disorder in a female patient accordin to claim 1, wherein the first dose of a beginning therapy is administered to a patient in the evening.

8. The method for treating a sexual disorder in a female patient according to claim 1, wherein the dose is administered in an amount of 1 to 100 mg.

9. The method for treating a sexual disorder in a female patient according to claim 8, the dose is 10 to 90 mg and is administered once daily or twice daily.

10. The method for treating a sexual disorder in a female patient according to claim 1, wherein the dose is administered once daily every evening.

11. (canceled)

12. The method for treating a sexual disorder in a female patient according to claim 1, wherein the sexual disorder is selected from the group consisting of Sexual Desire Disorders (such as Hypoactive Sexual Desire Disorders, Sexual Aversion Disorders, and Sexual Arousal Disorders), Orgasmic Disorders, Sexual Pain Disorders, Sexual Dysfunction due to a General Medical Condition, Substance-Induced Sexual Dysfunction, and Sexual Dysfunction not otherwise specified.

13. The method for treating a sexual disorder in a female patient according to claim 1, wherein the sexual disorder is Dyspareunia, Vaginismus, Noncoital Sexual Pain Disorder, Sexual Pain Disorder due to General Medical Condition, or Substance-induced Sexual Pain Disorder.

14. The method for treating a sexual disorder in a. female patient according to claim 1, wherein the sexual disorder is Sexual Desire Disorder, Hypoactive Sexual Desire Disorder (HSDD), Sexual Aversion Disorder, loss of sexual desire, lack of sexual desire, decreased sexual desire, inhibited sexual desire, loss of libido, libido disturbance, or frigidity.

15. The method for treating a sexual disorder) a female patient according to claim 1, wherein the sexual disorder is Hypoactive Sexual Desire Disorders and loss of sexual desire.

16. The method for treating a sexual disorder in a female patient according to claim 1, wherein the sexual disorder is selected from the group consisting of acquired Hypoactive Sexual Desire Disorder, acquired Sexual Aversion Disorder, acquired loss of sexual desire, acquired lack of sexual desire, acquired decreased sexual desire, acquired inhibited sexual desire, acquired loss of libido, acquired libido disturbance, and acquired frigidity.

17. The method for treating a sexual disorder in a female patient according to claim 1, wherein the sexual disorder is a post- or pre-menopausal Sexual Desire Disorder.

18. The method for treating a sexual disorder in a female patient according to claim 1, wherein the sexual disorder is Premenstrual Dysphoria, Premenstrual Syndrome, or Premenstrual Dysphoric Disorder.

19. The method for treating a sexual disorder in a female patient according to claim 1, wherein the medication is suitable for oral, rectal, or parenteral administration, or for nasal inhalation.

20. The method for treating a sexual disorder in a female patient according to claim 1, wherein the flibanserin derivative is a pharmacologically acceptable acid addition salt, or hydrate, or solvate of flibanserin.

21. The method for treating a sexual disorder in a female patient according to claim 1, wherein the medication comprises flibanserin.

22. The method for treating a sexual disorder in a female patient according to claim 21, wherein the medication comprises flibanserin as polymorph A of the free base, having a melting point of about 161° C. as measured using DSC.

23. The method for treating a sexual disorder in a female patient according to claim 1, wherein the pharmaceutically acceptable acid addition salt is selected from the salts formed by the acids selected from succinic acid, hydrobromic acid, acetic acid, fumaric acid, maleic acid, methanesulphonic acid, lactic acid, phosphoric acid, hydrochloric acid, sulphuric acid, tartaric acid, citric acid, and mixtures thereof.

24. The method for treating sexual disorder in a female patient according to claim 2, wherein the significant change of at least one primary criteria for efficacy and at least one secondary criteria for efficacy is determined over a period of time of one month or longer.

25. The method for treating sexual disorder in a female patient according to claim 2, wherein the medication to be administered to the female patient is adjusted to have a significant change in at least one of criteria a) or b), and at least one of criteria c), d), e), or f), to achieve the therapeutic most effective dose and/or most effective administration mode of the medication.

26. The method for treating sexual disorder in a female patient according to claim 3, wherein the medication to be administered to the female patient is adjusted to have a significant change in at least one of criteria a) or b), and at least one of criteria c), d), e), or f), to achieve the therapeutic most effective dose and/or most effective administration mode of the medication.

27. The method for treating a sexual disorder in a female patient according to claim 8, wherein the dose is administered in an amount of 1 to 40 mg.

28. The method for treating a sexual disorder in a female patient according to claim 8, wherein the dose is administered in an amount of 41 to 60 mg.

29. The method for treating a sexual disorder in a female patient according to claim 8, wherein the dose is administered in an amount of 61 to 90 mg.

30. The method for treating a sexual disorder in a female patient according to claim 8, wherein the dose is administered in an amount of 91 to 100 mg.
